(19) United States
(12) Patent Application Publication
(10) Pub. No.: US 2026/0097195 A1
(43) Pub. Date: Apr. 9, 2026

(54) MOTOR DRIVE CIRCUIT FOR A MECHANICAL CIRCULATORY SUPPORT DEVICE

(71) Applicant: Abiomed, Inc., Danvers, MA (US)

(72) Inventors: Hisham Hafez, Danvers, MA (US); Johannes Visser, Aachen (DE); Verena Zscherlich, Aachen (DE)

(21) Appl. No.: 19/348,458

(22) Filed: Oct. 2, 2025

Related U.S. Application Data
(60) Provisional application No. 63/703,584, filed on Oct. 4, 2024.

(51) Int. Cl. A61M 60/419; A61M 60/165; A61M 60/216; A61M 60/411

(52) U.S. Cl. CPC A61M 60/419; A61M 60/165; A61M 60/216; A61M 60/411; A61M 2205/103; A61M 2205/3365; A61M 2205/50

(57) ABSTRACT

Methods and apparatus for driving a motor of a mechanical circulatory support device are provided. The method includes generating a pulse width modulated (PWM) drive signal, selectively filtering the PWM drive signal driving a first motor phase having a high state, sensing a back electro-motive force (back EMF) signal induced in a second motor phase not being driven by the PWM drive signal, determining a rotor position based on the back EMF signal, and modifying the PWM drive signal based on the rotor position.

### CROSS REFERENCE TO RELATED APPLICATIONS

This application claims the benefit under 35 U.S.C. § 119(e) to U.S. Provisional Patent Application No. 63/703,584, filed October 4, 2024, and titled, “MOTOR DRIVE CIRCUIT FOR A MECHANICAL CIRCULATORY SUPPORT DEVICE,” the contents of which is incorporated by reference herein in its entirety.

### FIELD OF THE INVENTION

This disclosure relates a motor drive circuit for a mechanical circulatory support device.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Temporary cardiac support devices, such as heart pump systems (also referred to as “intracardiac blood pumps”), provide hemodynamic support and facilitate heart recovery. Intracardiac blood pumps have traditionally been used to temporarily assist the pumping function of a patient’s heart during emergent cardiac procedures, such as a stent placement, performed after the patient suffers a heart attack, cardiac arrest, and/or cardiogenic shock. Intracardiac blood pumps also may be used to take the load off of a patient’s heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient’s body via an elongate drive shaft (or drive cable) or by an onboard motor located inside the patient’s body. Examples of such devices include the Impella^{®} family of devices (Abiomed, Inc., Danvers, MA).

### SUMMARY

Described herein are systems and methods for controlling a motor (e.g., a permanent magnet synchronous motor (PMSM)), which may be used to drive operation of a mechanical circulatory support (MCS) device (e.g., a heart pump). Accurately determining the position of a rotor may be important to effectively and efficiently control the motor. For example, control circuitry for a synchronous motor in which the orientation of the magnetic field is changed as the rotor rotates may be configured to take the rotor position as input. In some such motors, an encoder may be used to directly measure the rotor position, which may be provided as feedback to the control circuitry to adjust the driving field accordingly. MCS devices may have a small form factor that precludes the use of an encoder to directly measure the rotor position during operation of the motor. The rotor position of such motors may be indirectly measured using a back electromotive force (back EMF) crossing technique in which a voltage induced from a first (energized) coil into a second (not energized) coil is measured. The inventors have recognized and appreciated that accurately obtaining such indirect measurements may be challenging when the motor drive signal output from the motor controller is a pulse width modulated (PWM) signal. To this end, some embodiments are directed to a motor drive circuit that includes filter circuitry configured to facilitate the indirect measurement of rotor position of a synchronous motor when a PWM signal is used as the motor drive signal.

In some embodiments, a drive circuit for a motor of a mechanical circulatory support device is provided. The drive circuit includes circuitry configured to output a pulse width modulated (PWM) drive signal, a filter circuit configured to filter the PWM drive signal to produce a filtered drive signal, an inverter configured to provide the filtered drive signal to a first phase of a three phase motor, a sensor configured to sense a back electro-motive force (back EMF) signal associated with a second phase of the motor when the first phase of the motor is driven with the filtered drive signal, and a controller. The controller is configured to determine a rotor position of the motor based, at least in part, on the back EMF signal, and modify the PWM drive signal based, at least in part, on the rotor position.

In one aspect, the drive circuit further includes a variable power supply configured to provide a supply voltage to the circuitry configured to output the PWM drive signal. In another aspect, the controller is configured to modify the PWM drive signal by setting a duty cycle of PWM drive signal based, at least in part, on the rotor position. In another aspect, the filter circuit comprises a low pass filter. In another aspect, the filter circuit comprises an inductor coupled to a capacitor. In another aspect, the inductor and the capacitor are coupled in series.

In another aspect, the motor is a three-phase motor including the first phase, the second phase and a third phase, and the controller is further configured to selectively enable the filter circuit for the first phase, the second phase or the third phase depending on which of the first phase, the second phase, or the third phase is associated with a high state in a six block commutation control scheme. In another aspect, the controller is further configured to selectively disable the filter circuit for any phase of the three-phase motor that is not associated with the high state.

In another aspect, the sensor comprises a voltage sensor. In another aspect, the controller is further configured to determine a slope of a portion of the back EMF signal, and determine a rotor position of the motor based, at least in part, on the slope of the portion of the back EMF signal. In another aspect, the portion of the back EMF signal comprises the portion of the back EMF signal during which the second phase is transitioning from a low state to a high state or from a high state to a low state.

In some embodiments, a mechanical circulatory support device is provided. The mechanical circulatory support device includes an impeller, a motor configured to drive rotation of the impeller at one or more speeds, and a drive circuit according to any of the drive circuits described herein.

In some embodiments, a method of driving a motor of a mechanical circulatory support device is provided. The method includes generating a pulse width modulated (PWM) drive signal, selectively filtering the PWM drive signal driving a first motor phase having a high state, sensing a back electro-motive force (back EMF) signal induced in a second motor phase not being driven by the PWM drive signal, determining a rotor position based on the back EMF signal, and modifying the PWM drive signal based on the rotor position.

In one aspect, generating the PWM drive signal comprises generating the PWM drive signal based, at least in part, on a variable supply voltage. In another aspect, modifying the PWM drive signal based on the rotor position comprises setting a duty cycle of the PWM drive signal based, at least in part, on the rotor position. In another aspect, selectively filtering the PWM drive signal comprises using a low pass filter to selectively filter the PWM drive signal. In another aspect, selectively filtering the PWM drive signal comprises filtering the PWM drive signal using an LC circuit.

In another aspect, the motor is a three-phase motor including the first motor phase, the second motor phase and a third motor phase, and selectively filtering the PWM drive signal comprises selectively filtering the PWM signal for the first motor phase, the second motor phase or the third motor phase depending on which of the first motor phase, the second motor phase, or the third motor phase is associated with a high state in a six block commutation control scheme. In another aspect, the method further includes selectively disabling filtering for any motor phase of the three-phase motor that is not associated with the high state. In another aspect, determining a rotor position based on the back EMF signal comprises determining a slope of a portion of the back EMF signal, and determining a rotor position of the motor based, at least in part, on the slope of the portion of the back EMF signal. In another aspect, the portion of the back EMF signal comprises the portion of the back EMF signal during which the second motor phase is transitioning from a low state to a high state or from a high state to a low state.

### BRIEF DESCRIPTION OF DRAWINGS

FIG. 1 shows an illustrative heart pump device that may be used, in accordance with some embodiments.

FIG. 2 shows a block diagram of a motor drive circuit, in accordance with some embodiments.

FIGS. 3A-3D schematically illustrate back electro-motive force (back EMF) signals sensed from a motor when pulse-width modulated (PWM) drive signals are used to drive the motor, in accordance with some embodiments.

FIGS. 3E-3H schematically illustrates back EMF signals sensed from a motor when pulse-amplitude modulated (PAM) drive signals are used to drive the motor, in accordance with some embodiments.

FIG. 4A illustrates a back EMF signal sensed from a motor when PWM drive signals are used to drive the motor, in accordance with some embodiments.

FIG. 4B illustrates a corresponding current signal associated with the back EMF signals shown in FIG. 4A.

FIG. 5A illustrates a first example of a back EMF signal sensed from a motor when a PWM drive signal has been filtered by a filter circuit, in accordance with some embodiments.

FIG. 5B illustrates a second example of a back EMF signal sensed from a motor when a PWM drive signal has been filtered by a filter circuit, in accordance with some embodiments.

FIG. 6A illustrates example hardware for controlling a three-phase motor, in accordance with some embodiments.

FIG. 6B illustrates a six block commutation sequence for controlling the three phases of the three-phase motor shown in FIG. 6A, in accordance with some embodiments.

FIG. 6C illustrates a filter circuit that may be used to selectively filter a PWM drive signal for one of the phases of a three-phase motor, in accordance with some embodiments.

FIG. 7 is a process for controlling a motor using a motor drive circuit, in accordance with some embodiments.

### DETAILED DESCRIPTION

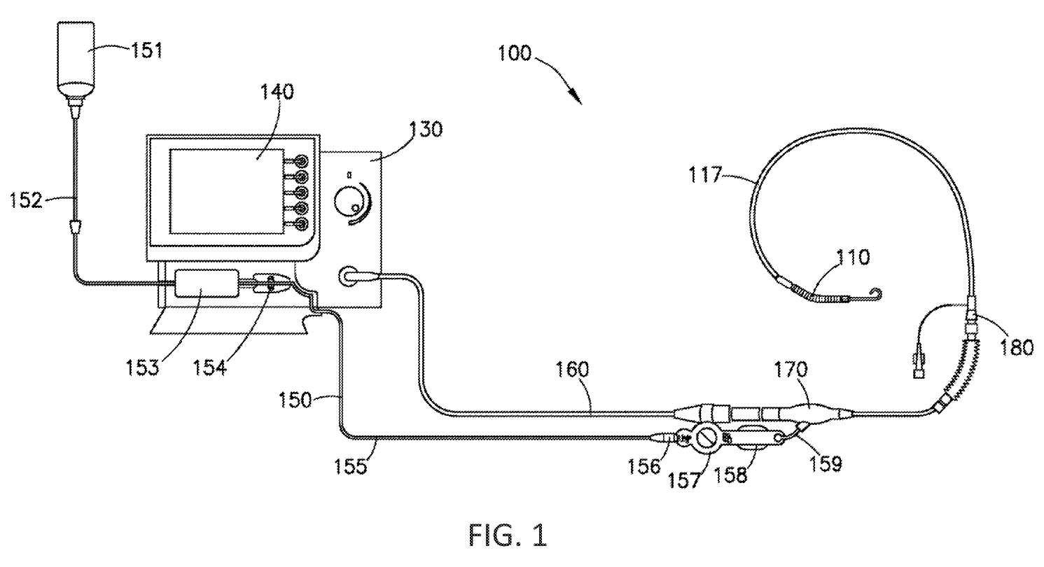

A circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller^{®}, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 may include a storage device (e.g., a memory) configured to store, for example, operating parameters to facilitate transfer of the patient to another controller if needed. Repositioning unit 180 may be used to reposition heart pump 110 in the patient’s heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. For example, controller 130 may be configured to control drive circuitry for the motor to modify how the motor is operating (e.g., by changing the speed and/or torque output of the motor as desired). As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor.

Various modifications can be made to cardiac support system 100 and one or more of its components. For instance, one or more additional sensors may be added to heart pump 100. In another example, a signal generator may be added to heart pump 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

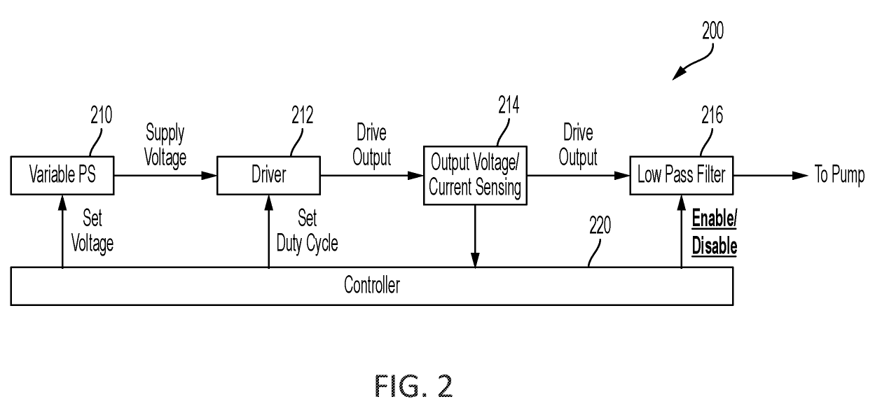

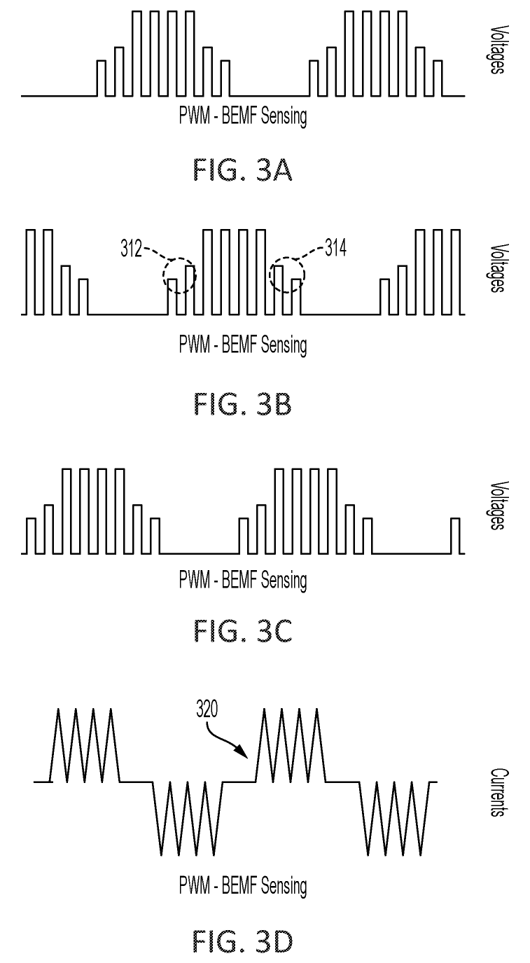

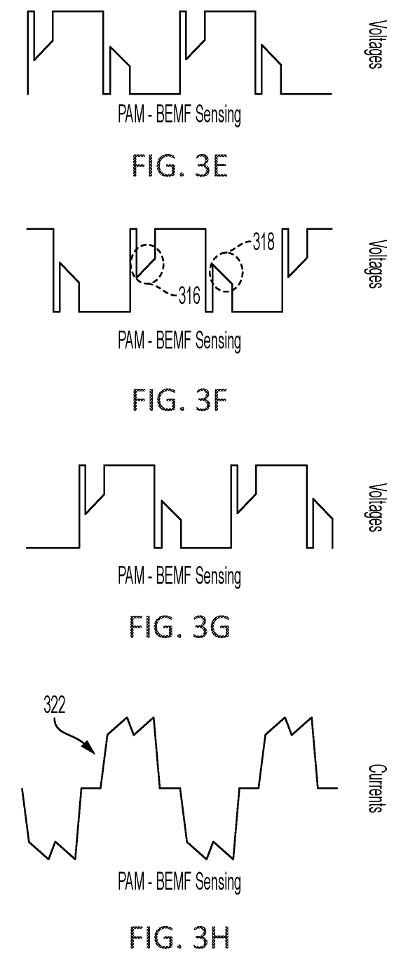

As described herein, a heart pump 110 may include a motor configured to drive rotation of an impeller that causes blood to flow from an inlet of the heart pump 110 to an outlet of the heart pump 110. Such a pumping action enables blood to be transported across one or more heart valves when the heart pump 110 is properly positioned within a patient’s heart. The motor may be driven by a motor drive circuit (also referred to herein simply as a “drive circuit”). FIG. 2 shows a block diagram of a drive circuit 200 that may be used to provide a drive signal to a motor, in accordance with some embodiments of the present disclosure. Drive circuit 200 may include a variable power source 210 configured to provide a supply voltage to driver circuitry 212. A controller 220 may be configured to provide a control signal to variable power source 210 to set the supply voltage based, for example, on a desired speed of the motor. Driver circuitry 212 may be configured to output a drive signal for driving the coils of the stator of the motor. In some embodiments, the motor may be a synchronous three-phase motor (e.g., a three-phase brushless DC motor), and the driver circuitry 212 may be configured to generate a pulse width modulated (PWM) drive signal that may be used to drive the stator coils associated with the three phases of the motor with a phase delay as shown in FIGS. 3A-3C. The controller 220 may be configured to provide a control signal to driver circuitry 212 to set the duty cycle of the drive signal output from the driver circuitry 212.

For a synchronous motor that includes permanent magnets, synchronization between the driving electrical field and the rotor position should be maintained to ensure proper and/or efficient operation of the motor. For instance, the rotor position should not lag behind the driving field by more than 90 degrees. Efficiency of the motor may be increased as the phase lag of the rotor position approaches, but does not exceed 90 degrees. A controller (e.g., controller 220) may be used to control the angle between the rotor position and the driving field. In particular, such a controller may take as input the rotor position and adjust the driving field (e.g., by modifying the drive signal(s)) as needed to achieve as close to a 90 degree phase lag as possible to improve motor efficiency.

As shown in FIG. 2, drive circuit 200 may include a sensing module 214 configured to sense a voltage or current associated with the drive signal output from the driver circuitry 212. For example, sensing module 214 may be configured to sense a back EMF signal induced in the coils of the motor. Motors used in some applications, such as mechanical circulatory support devices, may have a form factor that is small enough to ensure that the motor can be packaged within the mechanical circulatory support device. Because of the reduced form factor for such motors, inclusion of an encoder (e.g., a hall effect sensor) for directly sensing the position of the rotor during operation, may not be possible. Rather, such small form factor motor designs may rely on one or more sensors (e.g., sensing module 214) to indirectly determine or estimate the rotor position. One example type of indirect measurement is to measure the back EMF induced in coils associated with one or more phases of the motor that are not being driven by the drive signal (e.g., the PWM drive signal) of the drive circuit 200. As the magnets in the rotor of the motor rotate in the drive electric field, the rotation of the magnets induces a voltage in the motor windings (coils) of the motor, which may be referred to as an electro-motive force (EMF). The EMF induced in the coil gives rise to a secondary magnetic field that opposes the drive field for the motor, which is referred to as back EMF. The back EMF signal can be used to indirectly determine the rotor position, and the rotor position may be provided as feedback to the control circuitry outputting the PWM drive signal. In particular, the rotor position may be determined by examining a portion of the back EMF signal corresponding to time(s) when a particular phase of the motor is transitioning between excitation states (e.g., from a low state to a high state or a high state to a low state), also referred to herein as sensing a back EMF crossing.

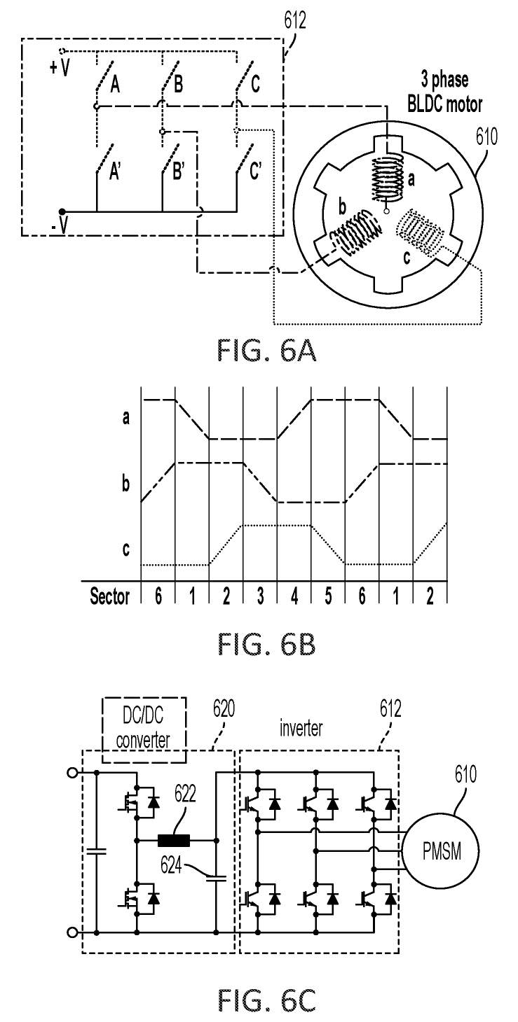

FIGS. 3A-3D schematically illustrate a PWM drive signal that may be generated to drive a three-phase synchronous motor, in accordance with some embodiments. FIGS. 3A-3C show that the back EMF voltage induced by the PWM drive signal has a trapezoidal shape when a PMW drive signal is used to drive each of the phases of the motor with six block commutation. For instance, phase A (FIG. 3A) of the motor may be driven with the PWM drive signal followed by phase B (FIG. 3B) with some time delay, followed by phase C (FIG. 3C) with some time delay, followed again by phase A (FIG. 3A) with some time delay, and so on. Six block commutation as a motor control technique is described in more detail in FIGS. 6A and 6B. As described herein, a technique for indirectly determining the rotor position is to examine a portion of the back EMF signal when a particular phase of the motor is transitioning between low and high states in the six block commutation control scheme. This can be observed in FIG. 3B as a first time period 312 during which phase B transitions from a low state to a high state and a second time period 314 during which phase B transitions from a high state to a low state. In some embodiments, the rotor position may be determined based, at least in part, on a slope of the back EMF signal sensed during such a time period.

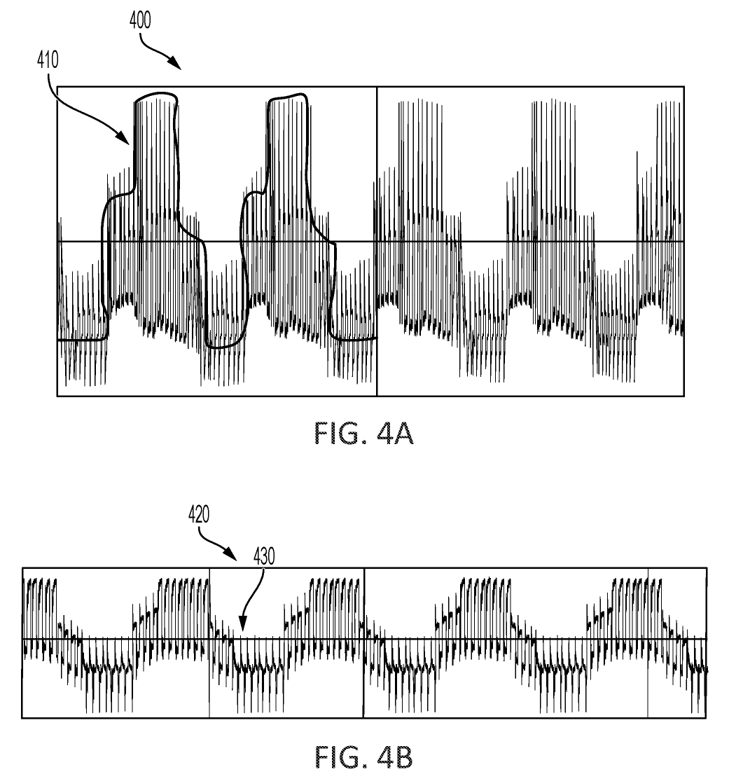

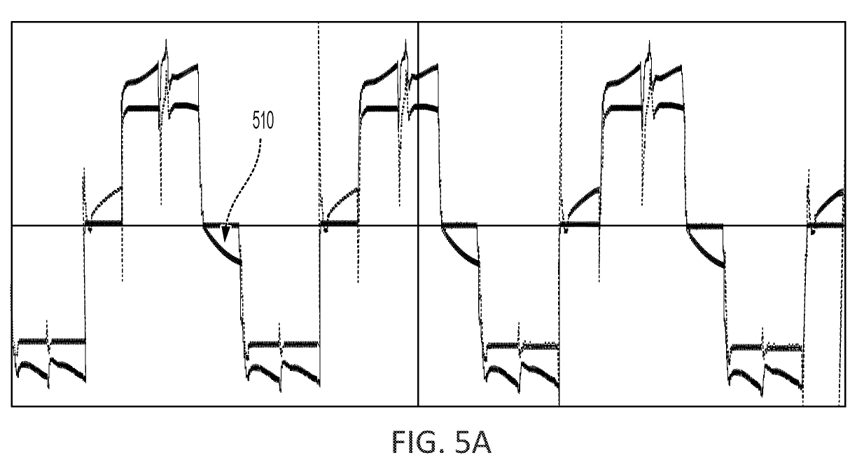

The corresponding current signal 320 for the voltage signals shown in FIGS. 3A-3C is shown in FIG. 3D. As shown, the use of a PWM signal with a low inductance motor with small coils induces current ripple or “spikes” in the current signal. This phenomenon can further be appreciated from the plots shown in FIGS. 4A and 4B. FIG. 4A illustrates a trapezoidal-shaped back EMF signal 410 that may be induced in coils of the motor when a PWM motor drive signal is used, in accordance with some embodiments. FIG. 4B illustrates the corresponding current signal 420 having a high current ripple introduced by the PWM drive signal. Such current ripple is particularly pronounced in motors with low inductance, which are often used in mechanical circulatory support devices. In particular, the current signal 420 includes current spikes that may make it challenging to accurately determine the slope of the back EMF signal during the time at which a phase is transitioning between low and high states. For instance, if a measurement of the back EMF signal is taken during one of the spikes, the magnitude of the signal value may not be representative of the slope of the signal at that point in time, resulting in an inaccurate measurement of the rotor position. By contrast, as shown in FIG. 3F, when the drive signal used to drive the motor is a pulse amplitude modulated (PAM) signal, the back EMF voltage signal shows a smooth slope during a first time period 316 during which phase B transitions from a low state to a high state and a second time period 318 when phase B transitions from a high state to a low state, which is also represented in the corresponding current signal 322 shown in FIG. 3H.

As can be appreciated from FIGS. 3A-3C, during six block commutation, a first phase of the motor is energized positively with the PWM drive signal (high state), a second phase of the motor is energized negatively with the PWM drive signal (low state), and the third phase of the motor is open or off. The inventors have recognized and appreciated that when a PMW drive signal is used, it may be advantageous to filter the PWM drive signal for only the phase that is currently set to a high state to reduce the spike contamination of the back EMF signal used for sensing the back EMF crossing and the rotor position determination.

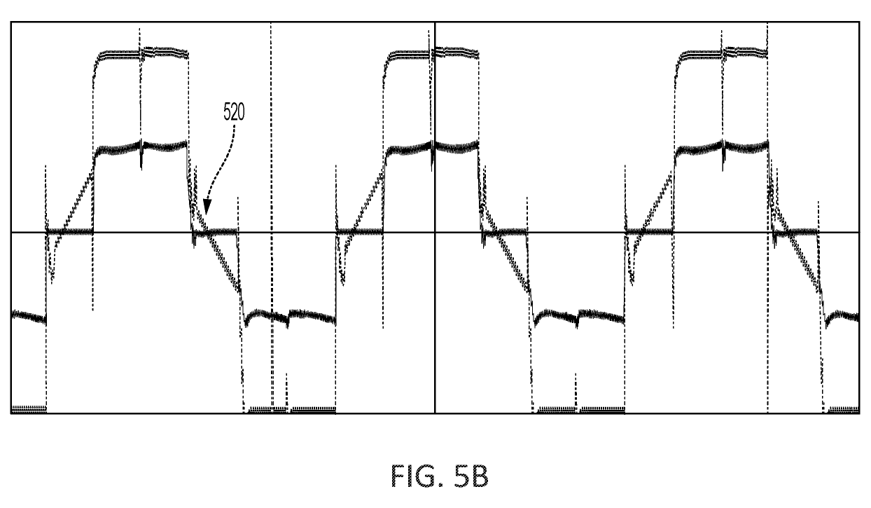

Returning to the drive circuit 200 shown in FIG. 2, drive circuit 200 may include a filter circuit 216 arranged to filter the PWM drive signal output from driver circuitry 212. In some embodiments, filter circuit 216 may be implemented as an LC circuit configured to provide low pass filtering of the input signal. A non-limiting example of a filter circuit 216 that may be used in accordance with some embodiments, is shown in FIG. 6C. As described herein, it may be advantageous to selectively apply the filter circuit to the PWM drive signal during rotation of the rotor. For instance, the filter circuit may be used to filter the PWM drive signal being provided to the phase currently associated with the high state in the six block commutation cycle. Controller 220 may be further configured to selectively enable or disable the filter circuit 216, such that the filter is applied only to the PWM drive signal used to drive the motor phase in a high state. FIGS. 5A and 5B show examples of selectively applying the filter circuit 216 to a PWM drive signal, in accordance with some embodiments. As shown in FIG. 5A, applying the filter circuit to the PWM drive signal of the phase having a high state resulted in a back EMF voltage signal 510 with reduced (or eliminated) spikes compared with the back EMF voltage signal shown in FIG. 4A. The corresponding current signal also shows less current ripple, which may improve the efficiency of the motor. By reducing or eliminating the spikes in the filtered PWM drive signal, the slope of the back EMF signal during the time when a phase is transitioning between low and high states may be more accurately determined. For instance, because there is less risk that the back EMF signal may be sampled during a spike, more samples can be considered (e.g., continuous sampling may be used) during the state transition time to determine the slope of the back EMF signal. By filtering the PWM drive signal in this way, the back EMF signal shown in FIGS. 3A-3D may more closely approximate the back EMF signal shown in FIGS. 3E-3H having reduced discrete switching noise compared with the signal shown in FIGS. 3A-3D. Such a reduced noise signal may enable a more accurate rotor position determination than when filtering is not used. As described herein, having an accurate rotor position determination using the techniques described herein may provide for more robust speed control of the motor. Additionally, reduction of the current ripple in the current signal may result in a more high efficiency motor operation and may generate less heat than conventional PWM driving techniques that do not include such a filter at the output of the motor drive signal.

FIG. 6A schematically shows hardware components used to control a three-phase brushless DC motor 610, in accordance with some embodiments. As shown, each of the phases a, b, and c are coupled to corresponding control circuit elements (e.g., semiconductor switches) in an inverter 612. The control circuit elements in inverter 612 are coupled to a high voltage +V and a low voltage -V, which may be ground. By controlling different control circuit elements to open and close at different timings, the three phases of the motors may be controlled to generate a corresponding electric field according to a desired control pattern by pulling any of the phases high or low. FIG. 6B shows plots corresponding to state changes for different phases of a motor when a six block commutation control scheme is used, in accordance with some embodiments. As shown, in a first sector, both phase a and phase b may be excited, with phase b being set to a high state and phase a being pulled from a high state to a low state. During the first sector, the filter circuit 216 may be enabled to filter the PWM signal being used to drive phase b, which is in the high state. In a second sector, phase a is switched off (e.g., by opening the switches A and Aˈ) and phase c is excited from a low state to a high state (e.g., by closing switch C), while phase b remains in the high state. In a third sector, phase c is now in the high state, while phase b is pulled to the low state (e.g., by closing switch Bˈ). During sector three, the filter circuit may be disabled for phase b, which is no longer in the high state and the filter circuit may be enabled for phase c, which is now in the high state. The process of enabling and disabling the filter circuit for the phase being excited in the high state may be repeated as the six block commutation cycle continues. By selectively enabling the filter circuit 216 only for the phase excited in the high state and measuring the back EMF signal in the phase that is off or open, a more accurate determination of the rotor position may be determined from the back EMF signal than if the filtering is not used.

FIG. 6C illustrates an example of a drive circuit for a motor 610 for a mechanical circulatory support device, in accordance with some embodiments. As shown in FIG. 6C, the drive circuit includes an inverter 612 configured to selectively couple the different phases of motor 610 to the drive signal in accordance with the six block commutator control sequence described in connection with FIG. 6A and 6B. The drive circuit shown in FIG. 6C further includes filter circuit 620, which includes an inductor 622 and a capacitor 624 coupled in series to form an LC circuit. It should be appreciated that any suitable LC circuit configured to perform low pass filtering of the PWM drive signal may be used, and the LC circuit shown in FIG. 6C is merely exemplary. As described herein, the filter circuit 620 may be configured to filter a PWM drive signal output from driver circuitry that generates a PWM drive signal based, at least in part, on information provided by a controller (e.g., controller 220 shown in FIG. 2). The controller may be further configured to enable filter circuit 620 to filter the PWM drive signal at a desired timing (e.g., when a particular phase is in a high state), examples of which are described herein.

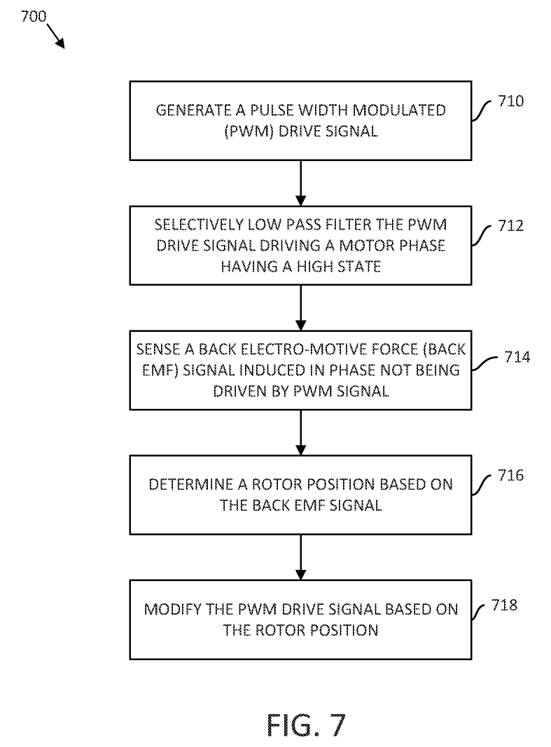

FIG. 7 is a flowchart of a process 700 for driving a motor for a mechanical circulatory support device, in accordance with some embodiments. Process 700 may begin in act 710, where a pulse width modulated (PWM) drive signal may be generated using, for example, driver circuitry 212 of drive circuit 200 shown in FIG. 2. Process 700 may proceed to act 712, where the PWM drive signal driving a motor phase having a high state may be selectively low pass filtered using, for example filter circuit 216 of drive circuit 200 shown in FIG. 2. Process 700 may then proceed to act 714, where a back electro-motive force (back EMF) signal induced in a phase not being driven by the PWM signal may be sensed. Process 700 may then proceed to act 716, where a rotor position may be determined based, at least in part, on the back EMF signal. Process 700 may then proceed to act 718, where the PWM drive signal may be modified based, at least in part, on the rotor position. For instance, a controller may modify a timing and/or amplitude of using the PWM drive signal to excite the phases of the motor such that the rotor position lags the rotating drive field by no more than 90 degrees.

Having thus described several aspects and embodiments of the technology set forth in the disclosure, it is to be appreciated that various alterations, modification, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A drive circuit for a motor of a mechanical circulatory support device, the drive circuit comprising:
circuitry configured to output a pulse width modulated (PWM) drive signal;
a filter circuit configured to filter the PWM drive signal to produce a filtered drive signal;
an inverter configured to provide the filtered drive signal to a first phase of a three phase motor;
a sensor configured to sense a back electro-motive force (back EMF) signal associated with a second phase of the motor when the first phase of the motor is driven with the filtered drive signal; and
a controller configured to:
determine a rotor position of the motor based, at least in part, on the back EMF signal; and
modify the PWM drive signal based, at least in part, on the rotor position.

2. The drive circuit of claim 1, further comprising:
a variable power supply configured to provide a supply voltage to the circuitry configured to output the PWM drive signal.

3. The drive circuit of claim 1, wherein the controller is configured to modify the PWM drive signal by setting a duty cycle of PWM drive signal based, at least in part, on the rotor position.

4. The drive circuit of claim 1, wherein the filter circuit comprises a low pass filter.

5. The drive circuit of claim 1, wherein the filter circuit comprises an inductor coupled to a capacitor.

6. The drive circuit of claim 5, wherein the inductor and the capacitor are coupled in series.

7. The drive circuit of claim 1, wherein
the motor is a three-phase motor including the first phase, the second phase and a third phase, and
the controller is further configured to selectively enable the filter circuit for the first phase, the second phase or the third phase depending on which of the first phase, the second phase, or the third phase is associated with a high state in a six block commutation control scheme.

8. The drive circuit of claim 7, wherein the controller is further configured to selectively disable the filter circuit for any phase of the three-phase motor that is not associated with the high state.

9. The drive circuit of claim 1, wherein the sensor comprises a voltage sensor.

10. The drive circuit of claim 1, wherein the controller is further configured to:
determine a slope of a portion of the back EMF signal, and
determine a rotor position of the motor based, at least in part, on the slope of the portion of the back EMF signal.

11. The drive circuit of claim 10, wherein the portion of the back EMF signal comprises the portion of the back EMF signal during which the second phase is transitioning from a low state to a high state or from a high state to a low state.

12. A mechanical circulatory support device, comprising:
an impeller;
a motor configured to drive rotation of the impeller at one or more speeds; and
a drive circuit according to claim 1.

13. A method of driving a motor of a mechanical circulatory support device, the method comprising:
generating a pulse width modulated (PWM) drive signal;
selectively filtering the PWM drive signal driving a first motor phase having a high state;
sensing a back electro-motive force (back EMF) signal induced in a second motor phase not being driven by the PWM drive signal;
determining a rotor position based on the back EMF signal; and
modifying the PWM drive signal based on the rotor position.

14. The method of claim 13, wherein generating the PWM drive signal comprises generating the PWM drive signal based, at least in part, on a variable supply voltage.

15. The method of claim 13, wherein modifying the PWM drive signal based on the rotor position comprises setting a duty cycle of the PWM drive signal based, at least in part, on the rotor position.

16. The method of claim 13, wherein selectively filtering the PWM drive signal comprises using a low pass filter to selectively filter the PWM drive signal.

17. The method of claim 13, wherein selectively filtering the PWM drive signal comprises filtering the PWM drive signal using an LC circuit.

18. The method of claim 13, wherein
the motor is a three-phase motor including the first motor phase, the second motor phase and a third motor phase, and
selectively filtering the PWM drive signal comprises selectively filtering the PWM signal for the first motor phase, the second motor phase or the third motor phase depending on which of the first motor phase, the second motor phase, or the third motor phase is associated with a high state in a six block commutation control scheme.

19. The method of claim 18, further comprising selectively disabling filtering for any motor phase of the three-phase motor that is not associated with the high state.

20. The method of claim 13, wherein determining a rotor position based on the back EMF signal comprises:
determining a slope of a portion of the back EMF signal, and
determining a rotor position of the motor based, at least in part, on the slope of the portion of the back EMF signal.

21. The method of claim 20, wherein the portion of the back EMF signal comprises the portion of the back EMF signal during which the second motor phase is transitioning from a low state to a high state or from a high state to a low state.
